(19) United States
(12) Patent Application Publication
(10) Pub. No.: US 2023/0218793 A1
(43) Pub. Date: Jul. 13, 2023

(54) DEVICE AND METHOD FOR CLEANING VEHICLE SURFACES

(71) Applicant: Gentherm GmbH, Odelzhausen (DE)

(72) Inventors: Rüdiger Spillner, Augsburg (DE); Chad Vincent Pacilli, Windsor (CA)

(21) Appl. No.: 18/017,120

(22) PCT Filed: Aug. 26, 2021
(86) PCT No.: PCT/US2021/047639

§ 371 Date: Jan. 20, 2023

Related U.S. Application Data
(60) Provisional application No. 63/073,525, filed on Sep. 2, 2020.

(51) Int. Cl. A61L 2/26; A61L 2/04; A61L 2/14; A61L 2/08; A61L 2/10; A61L 2/22; A61L 2/18; A61L 2/20; B60S 1/54; B60S 1/48

(52) U.S. Cl. CPC A61L 2/26; A61L 2/04; A61L 2/14; A61L 2/082; A61L 2/10; A61L 2/084; A61L 2/085; A61L 2/22; A61L 2/18; A61L 2/20; B60S 1/54; B60S 1/48; A61L 2202/17; A61L 2202/11; A61L 2202/25

(57) ABSTRACT

A purification device for cleaning one or more surfaces of a vehicle, the purification device configured to apply a purification process onto the one or more surfaces of the vehicle, the purification device includes a heating device for increasing a temperature of the surface.

### PRIORITY

This application claims priority to U.S. 63/073,525 filed on Sep. 2, 2020, the entirety of which is hereby incorporated by reference for all purposes.

### FIELD

These teachings relate to a device and method of cleaning, purifying, and/or disinfecting various vehicular surfaces.

### BACKGROUND

Certain cleaning and/or disinfecting methods may be applied to a soiled surface to clean, purify, and/or disinfect the surface, which may help reduce spread of disease and infection. However, certain cleaning and/or disinfecting methods are not easily applicable to vehicle surfaces. It may be desirable to improve the current state of the art by having a device and/or method for cleaning and/or disinfecting vehicle surfaces.

### SUMMARY

These teachings provide devices and methods for cleaning, purifying, and/or disinfecting various vehicular surfaces. The devices and methods disclosed herein may advantageously help reduce spread of disease and infection by cleaning, purifying, and/or disinfecting various vehicular surfaces. The vehicle surfaces may be those that are frequently or commonly contacted by one or more users or occupants and/or those that one or more users or occupants are nearby while entering, exiting, and/or inside the vehicle.

### BRIEF DESCRIPTION OF THE DRAWINGS

FIG. 1 is side view of a vehicle.

FIG. 2 is side view of a vehicle door of a vehicle.

FIG. 3 is side view of a vehicle door of a vehicle.

FIG. 4 is side view of a vehicle door of a vehicle.

FIG. 5 is side view of a vehicle door of a vehicle.

FIG. 6 is front view of a steering wheel of a vehicle.

FIG. 7 is side view of a vehicle seat of a vehicle.

### DETAILED DESCRIPTION

These teachings relate to one or more purification devices. The one or more purification devise may be one or more of any of the purification devices disclosed herein. The one or more purification devices may function to purify, clean, and/or disinfect one or more elements, assemblies, features, surfaces, of a vehicle. Purify, clean, and/or disinfect may mean that the purification device and/or the purification substance is operable to purify, clean, disinfect, and/or eliminate some, most or all soils, stains, germs, viruses, diseases that may be located on or within a surface. This may mean that the purification device and/or the purification substance is operable to purify, clean, disinfect, and/or eliminate 30% or more of the soils, stains, germs, viruses, diseases; 40% or more of the soils, stains, germs, viruses, diseases; 50% or more of the soils, stains, germs, viruses, diseases; 60% or more of the soils, stains, germs, viruses, diseases; 70% or more of the soils, stains, germs, viruses, diseases; 80% or more of the soils, stains, germs, viruses, diseases; 90% or more of the soils, stains, germs, viruses, diseases; 95% or more of the soils, stains, germs, viruses, diseases; 99% or more of the soils, stains, germs, viruses, diseases; etc.

The one or more elements, assemblies, features, surfaces of a vehicle may be located within the interior, passenger compartment of the vehicle. The one or more elements, assemblies, features, surfaces of a vehicle may be located on the exterior of the vehicle. The one or more elements, assemblies, features, surfaces of the vehicle may be ones that a vehicle occupant, passenger, and/or user may contact or come into close contact with. The one or more elements, assemblies, features, surfaces of the vehicle may be those that may become soiled with stains, disease, and/or infection. The one or more purification devices may function to clean or eliminate the stains, disease, and/or infection, which may advantageously provide a clean surface and/or help reduce spread of virus and disease.

The one or more purification devices may operate automatically or may self-activate. That is, for example, when a window or door is moved opened or rolled down, the one or more purification devices may function to purify, clean, and/or disinfect the window or surface thereof. The one or more purification devices may operate manually. That is, for example, a user may turn ON a purifying device when/as needed to purify, clean, and/or disinfect the window or surface thereof. The one or more purification devices may operate on a scheduled basis. For example, the one or more purification devices may be controlled and set to operate after a vehicle is turned ON or OFF, or after a predetermined amount of time has elapsed (for example after every 1 hour after 6 hours, after 12 hours, after 24 hours, etc.). The one or more purification devices may operate for a sufficient amount of time to clean, disinfect, and/or purify the surface. The amount of time may be less than 1 minute, 1 minute or more, 2 minutes or more, 3 minutes or more, 4 minutes or more, 5 minutes or more, 10 minutes or more.

While the one or more purification devices are operating, the surface may be locked or prevented from moving. For example, in the case of the surface being a window, during the purification process or method, the window may be located and prevented from being moved (up or down, side to side, etc.). On the other hand, while the one or more purification devices are operating, the surface of window may be moved or opened or closed.

The one or more purification devices may be at least partly imbedded in a window, a surface, a frame of a door or window, a vehicle seat, a dashboard, a door panel, an arm rest, a head rest, a steering wheel, button, knob, switch, etc. The one or more purification devices may be attached or applied to a surface of the surface of a frame of a door or window, a vehicle seat, a dashboard, a door panel, an arm rest, a head rest, a steering wheel, button, knob, switch, etc. The one or more purification devices may be positioned adjacent to a surface to be cleaned.

The one or more purification devices may be any of the one or more purification devices disclosed herein. The one or more purification devices may be used to apply one or more purification processes onto the one or more surfaces.

The one or more purification devices may be one or more radiant sources or devices configured for providing a purification substance onto or towards a surface to be cleaned, disinfected, and/or purified. The purification substance may be radiation, such as electromagnetic radiation, X-ray, light, microwave, IR, or infrared light; UV or ultraviolet light, or a combination thereof.

The one or more purification devices may be one or more heating devices, configured for increasing a temperature of a surface to be cleaned, disinfected, and/or purified beyond a certain or predetermined temperature. The purification substance may be an application of heat. During a heating application, the duration of the application of the purification substance or heat may be adapted or changed, depending on conditions such as an inside and/or outside temperature. For example, when the indoor and/or outdoor temperature is warmer, it may take less time for the heating device to raise the temperature of the surface to the predetermined temperature to clean, disinfect, and/or purify the surface. On the other hand, when the indoor and/or outdoor temperature is colder, it may take more time for the heating device to raise the temperature of the surface to the predetermined temperature to clean, disinfect, and/or purify the surface.

The heating device may comprise a PCT heater, a joule heating element, a heat pump, a Peltier device, a wire, a resistance wire, a heating wire, a foil, a conductive surface, a wire panel, or a combination thereof. The heating device may have fibers or an electrically conductive material. The heating device may have a thickness on the order of about 1 mm or more, 2 mm or more, 3 mm or more, 4 mm or more, 5 mm or more, etc.

The one or more heating devices may be heating strips. A heating strip may be a linear member applied to or embedded within the vehicle surface. The heating strip may be a meandering or curved strip applied to or embedded within a vehicle surface.

The heating device may have a power density that about 500 W/m² or more, 1000 W/m² or more, 1500 W/m² or more. The heating device may have one or more heat distribution layers configured for distributing the heat generated by heating device, across a desired portion of the surface. The heating device may be part of the vehicle HVAC system. For example, the heating device may be the vehicle ventilation heater, a seat heater, a steering wheel heater, or a combination thereof. The one or more heating devise may be different than the vehicle HVAC system (i.e., in addition to the vehicle HVAC system).

The one or more heating devices may be transparent or transparent. This may be advantageous if the heating device is attached to a window, glass, mirror, or other visible surface, such as a vehicle class-A surface.

The one or more heating devices or purification devices may be a member that is manually moved by an operator. For example, the heating device or purification device may be a sunshade or cover on the interior or cabin side of the surface.

The one or more purification devices may be one or more sanitizing devices, configured for applying or supplying one or more chemical substances on a surface to be cleaned, disinfected, and/or purified.

The one or more purification devices may be one or more plasma suppliers, configured for supplying a plasma or light-arc for treating a surface to be cleaned, disinfected, and/or purified with plasma or a light-arc.

The one or more purification devices may be a moveable member, such as a wiper made of cloth, rubber, sponge, or other suitable material. The moveable member may be a brush, a scrub brush, a squeegee,

The one or more purification devices may be a sprayer, mister, and/or sprinkler, configured for applying or supplying one or more chemical substances on a surface to be cleaned, disinfected, and/or purified.

The one or more purification devices may be a laser or light source configured for applying or supplying one or more lights, lasers, or optical waves on a surface to be cleaned, disinfected, and/or purified.

The one or more purification devices may be a stream of warm or hot air provided by the vehicle HVAC system.

The one or more purification devices may be or may include a fan or air mover, for moving or circulating the purification substance across or within the vehicle or vehicle cabin. The fan or air mover may have an inlet and outlet or shielding device on an inside or outside of the vehicle to keep a portion of the hot air or radiation from entering the rest of the interior of the vehicle or cabin. The shielding device may also function as a safety or control mechanism to act as a proximity sensor or touch sensor to shut down the purification process if touched by an object or occupant. The fan or air mover may remove air and/or the purification substance after the purification process or method has been completed.

The one or more purification devices may be a device that is configured to provide a substance that is activated, supported, suppressed, or controlled by the device. For example, the purification device may be one or more air ducts with one or more fans and with/without a heater to support purging the duct with hot air from the vehicle HVAC. For example, the purification device may be a heated surface that would increase chemical reaction or increase vaporization after treatment with chemical agents.

The vehicle, surface, and/or the one or more purification devices may include on more visual and/or audible indicators, configured for alerting a user or occupant of the status of the cleaning, purifying, and/or disinfecting process or method. For example, the one or more audible and/or visual indicators may indicate a need for cleaning, purifying, and/or disinfecting; completion of a cleaning, purifying, and/or disinfecting process; elapsed time of a cleaning, purifying, and/or disinfecting currently operating; and/or time remaining of a cleaning, purifying, and/or disinfecting process. The one or more indicators may provide an audible or visual indicator of a surface that has been cleaned, disinfected, and/or purified.

The one or more visual and/or audible indicators may be an LED light and/or a speaker, or screen with text or symbols. The one or more visual and/or audible indicators may provide information related to one or more parameters, such as: time since last purification or cleaning process; whether a purification or cleaning process is ON or operating and/or time left in a cleaning or purifying cycle; degree of pollution or amount of virus or germ detected; level of available purification substance remaining in a storage container or vessel; areas cleaned and/or remaining to be cleaned; or a combination thereof.

The vehicle, surface, and/or the one or more purification devices may include one or more safety control mechanisms or devices. A safety control device may detect human or occupant presence in a region to be cleaned; detect outside conditions before beginning a purifying process or method (i.e., checking for rain or snow before opening or closing a window; detect presence or absence of an occupant or object that may be pinched if a window is closed. The one or more safety control mechanisms or devices may function to provide a protection such that if a user or object comes into close proximity or touches the device, the purification process or device shut down to avoid injury to the object or occupant and/or damage to the surface, purification device, or both. The one or more safety control mechanisms or devices may shut down the device or process in the event a fault or malfunction of the process or device is detected, or a person or obstacle is near a surface to be treated.

The vehicle, surface, and/or the one or more purification devices may include one or more sensors. The one or more sensors may function to detect or evaluate when germs or viruses are present on a surface. The one or more sensors may evaluate a post-cleaning, purifying, and/or disinfecting process to determine if a second or subsequent cleaning, purifying, and/or disinfecting process is required to achieve a clean or germ-free surface.

The one or more purification devices may have one or more pollution detectors. A pollution detector may valuate one or more parameters, such as cleaning effect, efficiency, number of germs or pollution detected, cleaned, or to be cleaned; type of germ or virus present on the surface; an amount of purification substance remaining in a storage container or receptacle, or a combination thereof.

The one or more purification devices may be powered and/or controlled by a vehicle computer and/or processor. The one or more purification devices may be powered by a computer and/or processor that is different or in addition to the vehicle computer or processor.

The one or more purification devices may include one or more pumps, for moving the one or more purification substances from a storage tank or vessel to the surface to be cleaned.

The one or more purification devices may be use to apply or provide one or more purification substances onto any one or more surfaces of the vehicle. The one or more purification or cleaning substances may function to purify, clean, and/or disinfect, one or more vehicle surfaces. The one or more purification substances may reduce or eliminate diseases, viruses, dirt, debris, and/or contaminates. The one or more purification substances may reduce or eliminate a spread of diseases amongst passengers, occupants, and/or persons. This may be especially desirable when the one or more surfaces may be touched or contacted by one or more persons, for example when the vehicle or surface is acceptable by the public as may be the case in public transportation or ride sharing applications. The one or more purification substances may be one or more of any of the purification substances disclosed herein. The one or more purification substances may be a combination of two or more; three or more; four or more; etc. of any of the purification substances disclosed herein.

The one or more purification substances may be one or more of any of the purification substances disclosed herein. The one or more purification substances may include: warm, hot, ambient, or cold air; dry heat; detergents; lights for example UV light; radiation; electromagnetic radiation; chemicals; cleaning substances; plasma or light-arc. The one or more purification substances may be heat applied onto or directed to a surface. The one or more purification substances may be heat provided by a device that is imbedded within a surface. The one or more purification substances may be heat provided by a device that moves relative to the surface. The device may move automatically or manually relative to or along the surface.

In the case the purification substance is heat (i.e., the purification device is a heater or heating element), the heater may be configured to raise a temperature of the surface to a level sufficient to clean, purify, and/or disinfect the surface. The temperature may be 50° C. or more, 60° C. or more, 70° C. or more. 80° C. or more. In some instances, the temperature may be 56° C. or more. In some instances, the preferred temperature may be between 70-80° C. or more. The increase temperature alone may be sufficient to clean, disinfect, and/or purify the surface. In some configurations, however, it may be desirable to raise the surface temperature and also apply one or more additional purifying substances, such as a chemical, plasma, arc, etc.

In some configurations, the purification substance may be cold, or freezing air or surface. The cold or freezing surface may function to freeze the surface to clean, disinfect, and/or purify the surface.

The one or more purification substances may be a liquid, a gel, an aerosol, a gas, a chemically reactive gas, a solvent, or a combination thereof.

The one or more purification substances may be chlorine, soap, alcohol, a water-soluble compound, ethyl alcohol, isopropyl alcohol, methyl alcohol, hypochlorite, sodium hypochlorite, ethanol, water, bleach, sodium hypochlorite, Hydrogen peroxide, electrochemically activated (EA) water, or a combination thereof.

The one or more purification substances may be contained or stored in a solution tank or vessel. The purification device may also include one or more waste tanks or vessels for storing spent purification substances after cleaning, disinfecting, and/or purifying.

The one or more purification substances may be heat or conductive heat. For example, heat or conductive heat supplied by the one or more heating elements or devices may be the purification substance or may be used with one or more of the other purification substances disclosed herein. The heat may function to activate one or more of the purification substances disclosed herein. For example, a chemical reaction or vaporization may take place when heat is applied to one or more of the purification substances disclosed herein to activate the one or more purification substances.

In some configurations, one or more purification substances may activate one or more additional purification substances to achieve the purification outcome. For example, an application of heat or a heated surface may increase a chemical reaction or increase vaporization after the surface is treated with the purification substance. For example, a steering wheel or other touch surface may be treated with a purification substance, which may be activated after the a heating element inside of the steering wheel is activated or turned on. The warmth or heat form the heating element may activate the purification substance, which may function to clean and/or sanitize the steering wheel or surface. In another example, an air duct with a fan may include an optional heater to support purging of the duct with hot air from the vehicle HVAC.

The one or more purification devices and/or purification substances may function to clean, disinfect, and/or purify one or more germs, viruses, and/or soiled surfaces. The germs or viruses may be any germ or virus such as bacteria, viruses, fungi, protozoa, SARS-CoV-2, Covid-19, influenza,

The one or more purification devices and/or purification substances may function to clean, disinfect, and/or purify dirt, oil, debris, stains, smears, saliva, blood, urine, human waste, food, or a combination thereof. Any debris or stains tor residue, whether visible or invisible to the human eye, may be cleaned, purified, and/or disinfected with the purification device and/or substances disclosed herein.

The vehicle may be virtually any vehicle. For example, the vehicle may be a passenger car, truck, or utility vehicle. The vehicle may be a plane, train, boat, taxi, tram, sub way, etc. It is understood that these teachings are not limited to only vehicles. For example, these teachings may also be applied to non-vehicular applications, such as restaurants, office buildings, hotels, movie theaters, etc., where it may be desirable to purify, clean, and/or disinfect, one or more surfaces. Accordingly, one or more office, home, industrial, commercial, or other building windows, couches, chairs, recliners, counter tops, floors, etc. may be purified, cleaned, and/or disinfected with one or more of the purification devices and/or purification substances disclosed herein.

A vehicle surface may be any surface of the vehicle that a user or occupant may touch or come into close contact with. A vehicle surface may be located at or on the interior of the vehicle, inside the passenger compartment where a passenger is seated. A vehicle surface may be located at or on the exterior of the vehicle. The one or more surfaces or vehicle surfaces that may be cleaned, purified, and/or disinfected with the one or more purifying devices and/or purifying substances may be virtually any surface. For example, the one or more surfaces may be one or more windows, mirrors, consoles, seats, steering wheels, door panels, bolsters, door handles, radio or vehicle control knobs, switches, and buttons, seat belts, keys, key fobs, handles, headliners, cup holders, etc.

The one or more surfaces may be moved relative to the one or more purification devices and/or substances. For example, in the case the surface is a window, the window may be moved, slid, retracted, extended, tilted (i.e., up, and down and/or side to side) to guide the window or portion of the window along or through the purification device.

The one or more surfaces may be glass, plastic, metal, cloth, leather, wood, rubber, foam, textured, or a combination thereof.

### DETAILED DESCRIPTION OF PREFERRED EMBODIMENTS

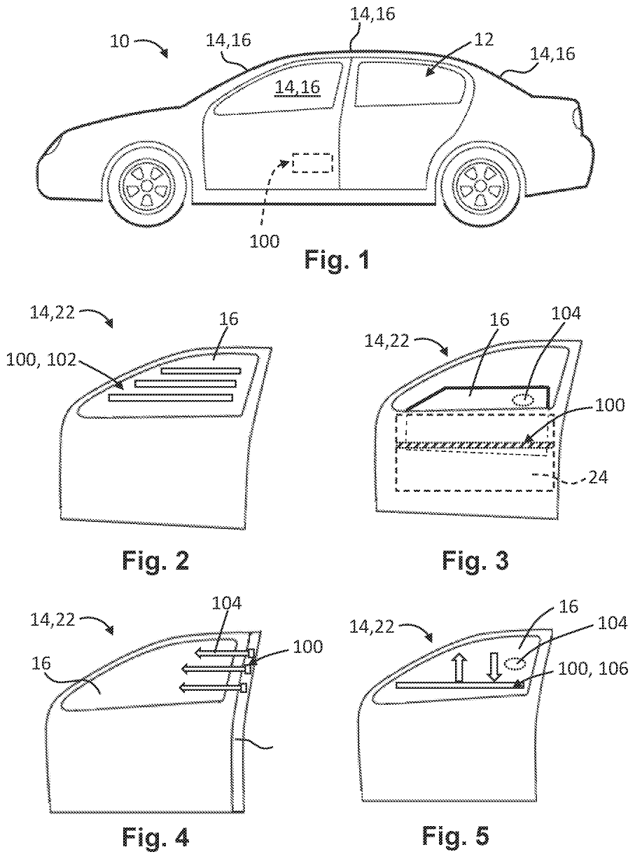

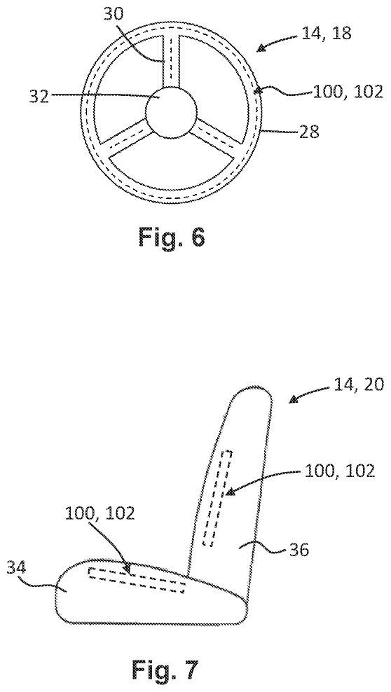

FIG. 1 illustrates a vehicle 10. The vehicle 10 comprises a passenger compartment 12. The passenger compartment 12 comprises one or more vehicle surfaces 14. A vehicle surface 14 may be any surface of the vehicle 12 that a user or occupant may touch or come into close contact with. For example, the one or more vehicle surfaces 14 may be a window 16 (front windshield, rear window, one or more side door windows, a sunroof, etc.), a steering wheel 18 (FIG. 6), a vehicle seat 20 (FIG. 7), a center console, a door panel, a door handle, a roof liner, a dashboard, an arm rest, a head rest, one or more buttons, switches, or knobs, etc.

The vehicle 10 and/or one or more of the vehicle surfaces 14 may comprise one or more purification devices 100. Additionally, or alternatively, the one or more air purification devices 100 may be discrete, stand-alone units that can be used with the vehicle 10 and/or the one or more vehicle surfaces 14 to purify, clean, and/or disinfect the vehicle surface 14.

FIG. 2 illustrates a vehicle door 22 of the vehicle 10 of FIG. 1. The vehicle door 22 has a window 16. A purification device 100 can be used to purify, clean, and/or disinfect the window 16. The purification device 100 may be any one or more of the purification devices 100 disclosed herein. The purification device 100 may be one or more heating elements 102. The one or more heating elements 102 may be applied on the window 16 (on the interior, passenger compartment side of the window 16 and/or on the exterior outside side of the window 16). The one or more heating elements 102 may be incorporated into the window 16 (i.e., provided within a thickness of the window 16). The one or more heating elements 102 may be transparent, translucent so as to not obstruct visibility through the window 16. Alternatively, the one or more heating elements 102 may be one or more thin wires that are visible on the window 16. The one or more heating elements 102 may be controlled to reach a predetermined temperature to purify, clean, and/or disinfect the window 16. It is understood that the one or more heating elements 102 can be located or used with any window 16 of the vehicle 10, including but not limited to: the windshield, rear window, sunroof, one or more front door windows, one or more back door windows, etc. The purification device 100 and/or heating elements 102 may also be used to purify, clean, and/or disinfect one or more vehicle mirrors, such as a rear-view mirror, one or more vanity mirrors located in a sun visor, side view mirrors, etc.

FIG. 3 illustrates a vehicle door 22 of the vehicle 10 of FIG. 1. The vehicle door 22 has a window 16. The window 16 may be moved or lowered from a raised position (i.e., FIG. 1 or 2 for example) to a lowered position or to an at least partially lowered position as illustrated in FIG. 3. The window 16 may be moved or lowered into a pocket or cavity 24 of the vehicle door 22. The pocket or cavity 24 may be located or defined within or behind a door panel of the vehicle door 22. A purification device 100 may be located within or adjacent to the pocket or cavity 24. The purification device 100 may be any one or more of the purification devices 100 disclosed herein.

During lowering of the window 16 into the cavity 24, the window 16 may pass by or pass through the one or more purification devices 100 which may purify, clean, and/or disinfect the window 16. For example, the one or more purification devices 100 may wipe or contact the window 16 while the window 16 is moved up and down. The one or more purification devices 100 may apply a purification substance 104 onto the window 16 while the window 16 moves up and/or down into or through the device 100. The purification substance 104 may be a suitable substance such as warm or hot air, a detergent, a spray, mist, or aerosol, an application of light, like UV light), etc. The one or more purification devices 100 may include one or more heating devices 102 located in the cavity 24. While the window 16 in FIG. 3 is illustrated as moving in a vertical direction (i.e., up, and down), it is understood that the window may instead be slid in a horizontal direction (i.e., front to back), tilted, etc. and still be subjected to one or more purification devices 100.

FIG. 4 illustrates a vehicle door 22 of the vehicle 10 of FIG. 1. The vehicle door 22 has a window 16. One or more purification devices 100 may be located in the vehicle 100 or vehicle door 22. The purification device 100 may be any one or more of the purification devices 100 disclosed herein. The purification device 100 may be located in a pillar or door frame 26 surrounding or supporting the door 22. The one or more purification devices 100 may apply a purification substance 104 onto the window 16, while the window 16 is in any position (i.e., completely up, completely down, or in a position in between. While the purification substance 104 is illustrated as being applied onto the window 16 from back to front, the purification substance 104 may instead be applied to the window 16 from front to back, top to bottom, bottom to top, or in any direction in between. The purification substance 104 may be warm or hot air, a detergent, a spray or mist, an application of light, like UV light), etc.

FIG. 5 illustrates a vehicle door 22 of the vehicle 10 of FIG. 1. The vehicle door 22 has a window 16. One or more purification devices 100 may be located in the vehicle 100 or vehicle door 22. The purification device 100 may be any one or more of the purification devices 100 disclosed herein. The purification device 100 may be a moveable member 106. The moveable member 106 may be configured to move relative to the window 16. The moveable member 106 may be configured to move up and down, side to side, or in any other direction, for example, diagonally, circular, etc.). The moveable member 106 may be configured to apply a purification substance 104 onto the window to purify, clean, and/or disinfect the window 16. The purification substance 104 may be warm or hot air, a detergent, a spray or mist, an application of light, like UV light), etc. The moveable member 106 may be a wiper.

FIG. 6 illustrates a steering wheel 18. The steering wheel 18 comprises a rim 28 and one or more spokes 30 extending between the rim 28 and a central portion or hub 32 of the steering wheel 18. One or more purification devices 100 may be located in or on the steering wheel 18, rim 28, and/or hub 32. The one or more purification devices 100 maybe any of the one or more purification devices disclosed herein. The one or more purification devices 100 may purify, clean, and/or disinfect the steering wheel 18. The one or more purification devices may be one or more heating elements 102.

FIG. 7 illustrates a vehicle seat 20. The vehicle seat 20 comprises a bottom or seating portion 34 and a back rest portion 36. One or more purification devices 100 may be located in or on the vehicle seat 20, the bottom or seating portion 34, and/or the back rest portion 36. The one or more purification devices 100 maybe any of the one or more purification devices disclosed herein. The one or more purification devices 100 may purify, clean, and/or disinfect the vehicle seat 20, the bottom or seating portion 34, and/or the back rest portion 36. The one or more purification devices may be one or more heating elements 102.

### LISTING OR REFERENCE NUMERALS

10 vehicle
12 passenger compartment
14 vehicle surfaces
16 window
18 steering wheel
20 vehicular seat
22 vehicular door
24 pocket or cavity
26 pillar or door frame
28 rim
30 spokes
32 hub
34 bottom, seating portion
36 back rest portion
100 purification device
102 heating elements
104 purification substance
106 moveable member

It is understood that the following method steps can be performed in virtually any order. Moreover, one or more of the following method steps can be combined with other steps; can be omitted or eliminated; can be repeated; and/or can separated into individual or additional steps.

The explanations and illustrations presented herein are intended to acquaint others skilled in the art with the invention, its principles, and its practical application. The above description is intended to be illustrative and not restrictive. Those skilled in the art may adapt and apply the invention in its numerous forms, as may be best suited to the requirements of a particular use.

Accordingly, the specific embodiments of the present invention as set forth are not intended as being exhaustive or limiting of the teachings. The scope of the teachings should, therefore, be determined not with reference to this description, but should instead be determined with reference to the appended claims, along with the full scope of equivalents to which such claims are entitled. The omission in the following claims of any aspect of subject matter that is disclosed herein is not a disclaimer of such subject matter, nor should it be regarded that the inventors did not consider such subject matter to be part of the disclosed inventive subject matter.

Plural elements or steps can be provided by a single integrated element or step. Alternatively, a single element or step might be divided into separate plural elements or steps.

The disclosure of “a” or “one” to describe an element or step is not intended to foreclose additional elements or steps. For example, disclosure of “a motor” does not limit the teachings to a single motor. Instead, for example, disclosure of “a motor” may include “one or more motors.”

While the terms first, second, third, etc., may be used herein to describe various elements, components, regions, layers and/or sections, these elements, components, regions, layers and/or sections should not be limited by these terms. These terms may be used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The invention illustratively disclosed herein suitably may be practiced in the absence of any element which is not specifically disclosed herein.

Any of the elements, components, regions, layers and/or sections disclosed herein are not necessarily limited to a single embodiment. Instead, any of the elements, components, regions, layers and/or sections disclosed herein may be substituted, combined, and/or modified with any of the elements, components, regions, layers and/or sections disclosed herein to form one or more embodiments that may be not be specifically illustrated or described herein.

The disclosures of all articles and references, including patent applications and publications, testing specifications, are incorporated by reference for all purposes. Other combinations are also possible as will be gleaned from the following claims, which are also hereby incorporated by reference into this written description.

1) A purification device for cleaning one or more surfaces of a vehicle, the purification device configured to apply a purification process onto the one or more surfaces of the vehicle, the purification device comprises a heating device for increasing a temperature of the surface.

2) The purification device according to claim 1, wherein the purification process includes controlling the heating device to:
a) heat the surface to a temperature between about 70-80° C.; and/or
b) heat or maintain a temperature of the surface for at least 5 min.

3) The purification device according to claim 1, wherein the purification process is applied to a window of the vehicle.

4) The purification device according to claim 1, wherein the heating device has fibers and/or a layer of electrically conductive material having a thickness of less than about 0.5 mm.

5) The purification device according to claim 1, wherein the purification device and the purification process further comprises at least one of the following:
a) a radiant device for providing electromagnetic radiation to the surface;
c) a sanitizer device for applying a chemical substance to the surface;
c) a plasma device for supplying a plasma and/or light-arc to the surface.

6) The purification device according to claim 5, wherein the purification device comprises at least two of a)-c).

7) The purification device according to claim 5, wherein the radiant device is controlled to provide least one of the following radiations: X-ray; light; microwave; infrared light; ultraviolet.

8) The purification device according to claim 1, wherein the purification device is a moveable member that moves relative to the surface of the vehicle.

9) The purification device according to claim 1, wherein the purification device is located within a cavity or pocket of a door panel.

10) The purification device according to claim 1, wherein the purification device is an air moving or blowing device.

11) The purification device according to claim 1, wherein the purification device and/or the purification process comprises:
a) one or more liquid sanitizers, ejected onto the surface as a liquid or aerosol;
b) one or more chemically reactive gases;
c) one or more liquid solvents; and/or
d) an additional purification substance that is activated, supported, suppressed, or controlled by the purification device.

12) The purification device according to claim 1, wherein the purification device is: at least partly imbedded in a frame of a door or window; and/or activated upon movement of the window or door.

13) The purification device according to claim 1, wherein the heating device is located within a thickness of the window.

14) The purification device according to claim 1, wherein the purification process includes controlling the heating device to heat the surface to a temperature between about 70-80° C. and heat or maintain a temperature of the surface for at least 5 minutes.

15) The purification device according to claim 1, wherein the purification device and the purification process further comprises:
a) a radiant device for providing electromagnetic radiation to the surface; and
c) a sanitizer device for applying a chemical substance to the surface; and
c) a plasma device for supplying a plasma and/or light-arc to the surface.

16) The purification device according to claim 3, wherein the purification device is a moveable member that moves relative to the surface of the vehicle.

17) The purification device according to claim 9, wherein the purification device is an air moving or blowing device.

18) The purification device according to claim 16, wherein the moveable member is a wiper, brush, or squeegee made of cloth, rubber, or sponge.

19) The purification device according to claim 18, wherein the purification device comprises a sprayer, mister, and/or sprinkler, configured for applying or supplying one or more chemical substances on the window.

20) The purification device according to claim 19, wherein the purification device is activated upon movement of the window or door.
